(19) United States
(12) Patent Application Publication
(10) Pub. No.: US 2025/0160678 A1
(43) Pub. Date: May 22, 2025

(54) GRAPHENE SENSOR SYSTEMS FOR CANNABINOID INGESTION ANALYSIS

(71) Applicant: VOCxI Health, Inc., Arden Hills, MN (US)

(72) Inventors: Gregory J. Sherwood, White Bear Lake, MN (US); Raia Colette Finc, Shoreview, MN (US); Gregory Kermit Peterson, Centerville, MN (US); Yung-Ping Yeh, Eden Prairie, MN (US); Randall Lee Schiestl, Brooklyn Park, MN (US)

(21) Appl. No.: 18/956,976

(22) Filed: Nov. 22, 2024

Related U.S. Application Data
(60) Provisional application No. 63/601,915, filed on Nov. 22, 2023.

(51) Int. Cl. A61B 5/08; G01N 27/22; G01N 33/497

(52) U.S. Cl. CPC A61B 5/082; G01N 27/226; G01N 33/4975

(57) ABSTRACT

Embodiments herein relate to systems and methods for detecting cannabinoid use and associated time frames of use. In an embodiment, a system for detecting cannabinoids in breath is included having a sensor device and a measurement circuit. The system can be configured to take measurements at a first time and at a second time and compare a pattern of the first time with a pattern of the second time and determine a time window of cannabinoid use based on the comparison. In an embodiment, a system for detecting cannabinoids in breath is included having a sensor device and a measurement circuit. The system can be configured to take measurements, match a pattern based on the measurements against a set of predetermined patterns, and determine a time window of cannabinoid use based on a match found amongst the set of predetermined patterns. Other embodiments are also included herein.

This application claims the benefit of U.S. Provisional Application No. 63/601,915, filed Nov. 22, 2023, the content of which is herein incorporated by reference in its entirety.

### FIELD

Embodiments herein relate to systems and methods for detecting cannabinoid use and associated time frames of use.

### BACKGROUND

Cannabinoids are molecules produced by the cannabis plant that interact with the body's natural Endocannabinoid System to regulate a variety of functions, such as inflammation, pain perception, mood, and memory. There are over 480 compounds in the cannabis plant, dozens of which are known as cannabinoids. Cannabinoids can regulate mood, blood sugar levels, appetite, treat nausea and suppress muscle spasms. Cannabinoids can have different purposes, such as being antidepressants, anti-inflammatory, anti-anxiety, anti-emetic, and even help protect the nervous system. Cannabinoids are also used recreationally.

Unfortunately, cannabinoid use (including THC—the main psychoactive compound in marijuana) can also acutely slow your reaction time, impair judgment of distance, and decrease coordination, all of which are essential to safely operating equipment, such as a vehicle.

### SUMMARY

Embodiments herein relate to systems and methods for detecting cannabinoid use and associated time frames of use. In a first aspect, a system for detecting cannabinoids in breath can be included having a sensor device, and a measurement circuit. The system can be configured to take measurements at a first time and at a second time, and compare data of the first time with data of the second time and determine a time window of cannabinoid use based on the comparison.
In a second aspect, in addition to one or more of the preceding or following aspects, or in the alternative to some aspects, the system can be configured to compare a data pattern of the first time with a data pattern of the second time and determine a time window of cannabinoid use based on the comparison.
In a third aspect, in addition to one or more of the preceding or following aspects, or in the alternative to some aspects, the measurement circuit can be configured to provide a voltage stimulus to the sensor device and measure resulting capacitance values of the graphene sensor elements.
In a fourth aspect, in addition to one or more of the preceding or following aspects, or in the alternative to some aspects, the voltage stimulus can be provided over a range of voltage values.
In a fifth aspect, in addition to one or more of the preceding or following aspects, or in the alternative to some aspects, the sensor device can include a first set of graphene sensor elements and a second set of graphene sensor elements.
In a sixth aspect, in addition to one or more of the preceding or following aspects, or in the alternative to some aspects, the first set of graphene sensor elements can include graphene varactors and the second set of graphene sensor elements can include the graphene varactors.
In a seventh aspect, in addition to one or more of the preceding or following aspects, or in the alternative to some aspects, the sensor device can include a graphene element and a compound (or sensitizing compound) can be disposed on a surface of the graphene element.
In an eighth aspect, in addition to one or more of the preceding or following aspects, or in the alternative to some aspects, the compound can include at least one selected from the group consisting of pyrenes, coronenes, aromatic cyclodextrins, and pillarenes.
In a ninth aspect, in addition to one or more of the preceding or following aspects, or in the alternative to some aspects, the compound can be in the form of a self-assembling monolayer.
In a tenth aspect, in addition to one or more of the preceding or following aspects, or in the alternative to some aspects, the data reflect amounts of cannabinoid and/or cannabinoid metabolites.
In an eleventh aspect, in addition to one or more of the preceding or following aspects, or in the alternative to some aspects, the data reflect relative amounts of cannabinoid and/or cannabinoid metabolites.
In a twelfth aspect, in addition to one or more of the preceding or following aspects, or in the alternative to some aspects, the data reflect amounts of cannabinoids and/or cannabinoid metabolites with different half-lives in vivo.
In a thirteenth aspect, in addition to one or more of the preceding or following aspects, or in the alternative to some aspects, the cannabinoids and/or cannabinoid metabolites can include at least one selected from the group consisting of delta-9-tetrahydrocannabinol (THC), cannabidiol (CBD), 11-OH-THC, 11-COOH-THC, 7-OH-CBD, and 7-COOH-CBD.
In a fourteenth aspect, in addition to one or more of the preceding or following aspects, or in the alternative to some aspects, the system can be configured to estimate cannabinoid and/or cannabinoid metabolite amounts.
In a fifteenth aspect, a method of detecting cannabinoid ingestion timing can be included. The method can include taking measurements using a graphene sensor at a first time and at a second time, comparing data of the first time with data of the second time, and determining a time window of cannabinoid use based on the comparison.
In a sixteenth aspect, in addition to one or more of the preceding or following aspects, or in the alternative to some aspects, the method can further include providing a voltage stimulus to the graphene sensor and measuring resulting capacitance values of the graphene sensor elements.
In a seventeenth aspect, in addition to one or more of the preceding or following aspects, or in the alternative to some aspects, the method can further include providing a voltage stimulus to the graphene sensor over a range of voltage values and measuring resulting capacitance values of the graphene sensor elements.
In an eighteenth aspect, in addition to one or more of the preceding or following aspects, or in the alternative to some aspects, the method can further include estimating cannabinoid and/or cannabinoid metabolite amounts.
In a nineteenth aspect, in addition to one or more of the preceding or following aspects, or in the alternative to some aspects, the method can further include taking breath samples of a test subject at a first time and at a second time.
In a twentieth aspect, in addition to one or more of the preceding or following aspects, or in the alternative to some aspects, the graphene sensor can be a graphene varactor.
In a twenty-first aspect, in addition to one or more of the preceding or following aspects, or in the alternative to some aspects, the graphene sensor can include a graphene element. A compound can be disposed on a surface of the graphene element.
In a twenty-second aspect, in addition to one or more of the preceding or following aspects, or in the alternative to some aspects, the compound can be in the form of a self-assembling monolayer.
In a twenty-third aspect, in addition to one or more of the preceding or following aspects, or in the alternative to some aspects, the compound can include at least one selected from the group consisting of pyrenes, coronenes, aromatic cyclodextrins, and pillarenes.
In a twenty-fourth aspect, a system for detecting cannabinoids in breath can be included having a sensor device and a measurement circuit. The system can be configured to take measurements to generate sample data, analyze the sample data, and determine a time window of cannabinoid use based on the analysis.
In a twenty-fifth aspect, in addition to one or more of the preceding or following aspects, or in the alternative to some aspects, the analysis includes matching a pattern based on the measurements against a set of predetermined patterns.
In a twenty-sixth aspect, in addition to one or more of the preceding or following aspects, or in the alternative to some aspects, the analysis includes interpolation to find a non-nodal point within an elimination curve representing a time since cannabinoid ingestion.
In a twenty-seventh aspect, in addition to one or more of the preceding or following aspects, or in the alternative to some aspects, the analysis includes calculating a time since cannabinoid ingestion based on a present amount of at least two cannabinoids and half-lives thereof.
In a twenty-eighth aspect, in addition to one or more of the preceding or following aspects, or in the alternative to some aspects, the analysis includes calculating a time since cannabinoid ingestion based on a present amount of at least one cannabinoid, an estimated starting amount of at least one cannabinoid, and a half-life of the at least one cannabinoid.
In a twenty-ninth aspect, in addition to one or more of the preceding or following aspects, or in the alternative to some aspects, the measurement circuit can be configured to provide a voltage stimulus to the sensor device and measure resulting capacitance values of the graphene sensor elements.
In a thirtieth aspect, in addition to one or more of the preceding or following aspects, or in the alternative to some aspects, the voltage stimulus can be provided over a range of voltage values.
In a thirty-first aspect, in addition to one or more of the preceding or following aspects, or in the alternative to some aspects, the sensor device can include a set of graphene sensor elements.
In a thirty-second aspect, in addition to one or more of the preceding or following aspects, or in the alternative to some aspects, the set of graphene sensor elements can include graphene varactors.
In a thirty-third aspect, in addition to one or more of the preceding or following aspects, or in the alternative to some aspects, the sensor device can include a graphene element and a compound (or sensitizing compound) can be disposed on a surface of the graphene element.
In a thirty-fourth aspect, in addition to one or more of the preceding or following aspects, or in the alternative to some aspects, the compound can include at least one selected from the group consisting of pyrenes, coronenes, aromatic cyclodextrins, and pillarenes.
In a thirty-fifth aspect, in addition to one or more of the preceding or following aspects, or in the alternative to some aspects, the compound can be in the form of a self-assembling monolayer.
In a thirty-sixth aspect, in addition to one or more of the preceding or following aspects, or in the alternative to some aspects, the sample data reflect amounts of cannabinoid and/or cannabinoid metabolites.
In a thirty-seventh aspect, in addition to one or more of the preceding or following aspects, or in the alternative to some aspects, the sample data reflect relative amounts of cannabinoid and/or cannabinoid metabolites.
In a thirty-eighth aspect, in addition to one or more of the preceding or following aspects, or in the alternative to some aspects, the sample data reflect amounts of cannabinoids and/or cannabinoid metabolites with different half-lives in vivo.
In a thirty-ninth aspect, in addition to one or more of the preceding or following aspects, or in the alternative to some aspects, the cannabinoids and/or cannabinoid metabolites can include at least one selected from the group consisting of delta-9-tetrahydrocannabinol (THC), cannabidiol (CBD), 11-OH-THC, 11-COOH-THC, 7-OH-CBD, and 7-COOH-CBD.
In a fortieth aspect, in addition to one or more of the preceding or following aspects, or in the alternative to some aspects, the system can be configured to estimate cannabinoid and/or cannabinoid metabolite amounts.
In a forty-first aspect, a method of detecting cannabinoid ingestion timing can be included. The method can include taking measurements using a graphene sensor, analyzing the measurements, and determining a time window of cannabinoid use based on the analysis.
In a forty-second aspect, in addition to one or more of the preceding or following aspects, or in the alternative to some aspects, the method further can include matching a pattern based on the measurements against a set of predetermined patterns and determining a time window of cannabinoid use based on a match found amongst the set of predetermined patterns.
In a forty-third aspect, in addition to one or more of the preceding or following aspects, or in the alternative to some aspects, the method can further include interpolating to find a non-nodal point within an elimination curve representing a time since cannabinoid ingestion.
In a forty-fourth aspect, in addition to one or more of the preceding or following aspects, or in the alternative to some aspects, the method can further include calculating a time since cannabinoid ingestion based on a present amount of at least two cannabinoids and half-lives thereof.
In a forty-fifth aspect, in addition to one or more of the preceding or following aspects, or in the alternative to some aspects, the method can further include calculating a time since cannabinoid ingestion based on a present amount of at least one cannabinoid, an estimated starting amount of at least one cannabinoid, and a half-life of the at least one cannabinoid.
In a forty-sixth aspect, in addition to one or more of the preceding or following aspects, or in the alternative to some aspects, the method can further include providing a voltage stimulus to the sensor device and measure resulting capacitance values of the graphene sensor elements.
In a forty-seventh aspect, in addition to one or more of the preceding or following aspects, or in the alternative to some aspects, the method can further include providing a voltage stimulus to the sensor device over a range of voltages and measuring resulting capacitance values of the graphene sensor elements.
In a forty-eighth aspect, in addition to one or more of the preceding or following aspects, or in the alternative to some aspects, the method can further include estimating cannabinoid and/or cannabinoid metabolite amounts.
In a forty-ninth aspect, in addition to one or more of the preceding or following aspects, or in the alternative to some aspects, the method can further include taking breath samples of a test subject.
In a fiftieth aspect, in addition to one or more of the preceding or following aspects, or in the alternative to some aspects, the graphene sensor can be a graphene varactor.
In a fifty-first aspect, in addition to one or more of the preceding or following aspects, or in the alternative to some aspects, the graphene sensor can include a graphene element, wherein a compound can be disposed on a surface of the graphene element.
In a fifty-second aspect, in addition to one or more of the preceding or following aspects, or in the alternative to some aspects, the compound can be in the form of a self-assembling monolayer.
In a fifty-third aspect, in addition to one or more of the preceding or following aspects, or in the alternative to some aspects, the compound can include at least one selected from the group consisting of pyrenes, coronenes, aromatic cyclodextrins, and pillarenes.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a chart showing elimination curves of various cannabinoids.

FIG. 2 is a chart showing elimination curves of various cannabinoids.

FIG. 3 is a chart showing elimination curves of various cannabinoids and a reference compound.

FIG. 4 is a schematic view of various components of a system in accordance with various embodiments herein.

FIG. 5 is a schematic top plan view of a chemical sensor element in accordance with various embodiments herein.

FIG. 6 is a schematic diagram of a portion of a measurement zone in accordance with various embodiments herein.

FIG. 7 is a schematic perspective view of a graphene varactor in accordance with various embodiments herein.

FIG. 8 is a schematic cross-sectional view of a portion of a graphene varactor in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Since some cannabinoids and/or metabolites thereof break down relatively slowly in the body, it can be difficult to detect whether an individual has ingested a psychoactive cannabinoid within a time frame of relevance for safety purposes. For example, it can be difficult to determine whether an individual has ingested a cannabinoid recently enough to affect their ability to safely operate equipment such as a vehicle.

Cannabinoids and/or metabolites thereof exhibit different physiological half-lives. Because of this, the relative amounts of one compound to another changes over time after ingestion in a characteristic way. Systems herein can be used to detect patterns of compounds, including cannabinoids and/or metabolites thereof. Because the relative amounts of one compound to another change as a function of time after ingestion, the patterns detected by systems herein also change as a function of time after ingestion. Therefore, embodiments of systems herein can be used to detect patterns that can then be used to work backward to identify a likely time window of cannabinoid ingestion. In some cases, detected patterns can also be influenced by the relative amounts of cannabinoids present. As such, embodiments of systems herein can also be used to identify amounts of cannabinoids that are present.

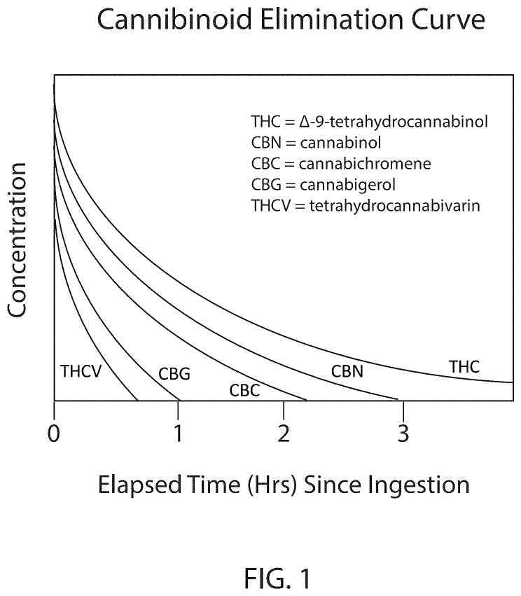

Referring now to FIG. 1, a chart is shown of elimination curves of various cannabinoids. As can be seen, the elimination curves are different for each compound based on the fact that they each have different half-lives in-vivo. In specific, FIG. 1 shows elimination curves for delta-9-tetrahydrocannabinol (THC), cannabinol (CBN), cannabichromene (CBC), cannabigerol (CBG), and tetrahydrocannabivarin (THCV). As can be seen, the relative amounts of one compound to another vary with time after an ingestion event. The relative amounts of cannabinoid metabolites (e.g., intermediate or final breakdown products of cannabinoid compounds in the body) will also vary with time after an ingestion event.

Systems herein can include chemical sensor elements that include sensitivity to compounds such as cannabinoids and their metabolites. In some embodiments, chemical sensor elements can be configured to bind one or more analytes, such as volatile organic compounds (VOCs), in a complex sample mixture, such as a gaseous sample mixture. The chemical sensor elements can include graphene-based sensors. For example, the chemical sensor elements can include graphene-based quantum capacitance varactors (“graphene varactors”) that can exhibit a change in capacitance in response to an applied voltage as a result of the presence of one or more analytes, such as volatile organic compounds (VOCs) on a surface of the graphene varactor. In this way, gas samples can be analyzed by contacting them with a graphene varactor-based sensor element, providing a bias voltage, and measuring capacitance or voltage values. In many cases, the graphene varactor-based sensor elements can be exposed to a range of bias voltages to discern features such as the Dirac point (or the bias voltage at which the varactor exhibits the lowest capacitance). The response signal generated by the discrete binding detectors in the presence or absence of one or more analytes can be used to characterize the content of the gaseous mixture. As such, each gaseous mixture can exhibit a unique set of response signals, or “fingerprint,” for any given array.

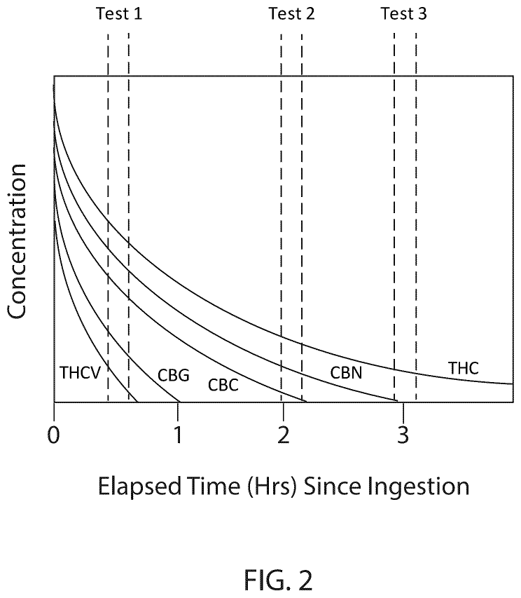

As previously referenced, the relative amounts of one cannabinoid compound to another and/or metabolites thereof will vary with time after an ingestion event because their different half-lives in the body. As such, a pattern of compounds captured with a system herein at time point that is closer to the time of ingestion will be different than a pattern of compounds captured at a later time. Referring now to FIG. 2, a chart is shown of elimination curves of various cannabinoids. As with FIG. 1, FIG. 2 shows elimination curves for delta-9-tetrahydrocannabinol (THC), cannabinol (CBN), cannabichromene (CBC), cannabigerol (CBG), and tetrahydrocannabivarin (THCV). In this example, exemplary test times are shown in the form of Test 1, Test 2, and Test 3, with each test being progressively spaced out farther away in time from the time of ingestion. As can be seen, relative amounts of the exemplary cannabinoids shown varies at each test time. Because of this, patterns as identified by systems herein are different at each test time. For example, at the time of Test 1 (which may be a half-hour after cannabinoid ingestion) the amount of CBN is roughly 80 to 90 percent of the amount of THC. In contrast, at the time of Test 2 (which may be two hours after cannabinoid ingestion) the amount of CBN is only about 50 percent of the amount of THC. As such, the relative amounts of these compounds has changed substantially (in a time-dependent manner based on different half-lives) and this is reflected in data generated by systems herein.

Systems herein can capture data reflecting patterns of cannabinoid compounds and/or their metabolites in the breath of an individual and then use the same to determine a likely time window of cannabinoid ingestion. The system can accomplish this in various ways as described further herein. For example, in one approach the system can match a sample pattern gathered from the breath of an individual against a set of template patterns, wherein the template patterns are indexed by time to reflect characteristic patterns of the cannabinoid compounds and/or metabolites at different time points after ingestion. The template pattern that represents the best fit can then be used to estimate the time of cannabinoid ingestion. For example, a template pattern that is characteristic of use in the preceding 60 to 90 minutes, if matched, can be used by the system to determine that cannabinoid ingestion likely occurred in the preceding 60 to 90 minutes.

In another approach, the system can use the relative amounts or ratios of different cannabinoid compounds directly to estimate a time of ingestion. For example, using a standard elimination curve (such as that shown in FIG. 1 or FIG. 2), a ratio of cannabinoid compounds in a breath sample can be matched against the elimination curves to determine what time point on the elimination curve is the best fit for the breath sample and that matching time point can be used as an estimate of the amount of time that has elapsed since cannabinoid ingestion. For example, an interpolation approach can be used to find a non-nodal point within an elimination curve representing a time since cannabinoid ingestion.

In another approach, the half-lives of specific cannabinoids can be used to determine an estimated time of ingestion. The following formula I described an amount estimated at time N(t) based upon a starting amount N(0), a half-life (T), and elapsed time (t):

N(t)=N(0)×0.5(t/T) Formula I

As such, the ratio of two compounds (A and B) can be calculated according to formula II using the output of formula I:

NA(t):NB(t) Formula II

Thus, the elapsed time (t) can be calculated by taking an observed ratio of two compounds, assuming a starting ratio of the two compounds at time 0 (based on known relative amounts of the compounds in the ingested substance), and then working backwards to get to an elapsed time (t). As such, in some embodiments the system can be configured to calculate a time since cannabinoid ingestion based on a present amount of at least two cannabinoids and half-lives thereof. In some embodiments, the system can be configured to calculate a time since cannabinoid ingestion based on a present amount of at least one cannabinoid, an estimated starting amount of at least one cannabinoid, and a half-life of the at least one cannabinoid.

In some embodiments, systems herein can be configured to use the results of testing at a single point in time to determine an estimated time of substance ingestion. However, instead or in addition, in some embodiments systems herein can be configured to use the results of testing at two or more discrete time points to aid in estimating a time of ingestion. For example, with knowledge of the expected changes in concentrations of various cannabinoids and/or metabolites thereof due to varying half-lives, specific changes would be expected to occur in terms of relative amounts of cannabinoid compounds and/or metabolites between a first time and a second time. By evaluating a change in relative amounts of cannabinoids, metabolite thereof, and/or patterns representing the same, an estimated time of ingestion can be determined.

In some embodiments, a reference compound can be evaluated to set a baseline for detected cannabinoid compounds and metabolites and/or to aid in estimate amounts of cannabinoid compounds and/or metabolites thereof present in a sample, such as a breath sample of an individual. For example, a physiological compound that is known to be present in breath at a consistent level can be used as a reference compound. For example, acetaldehyde is known to be present in the breath of healthy individuals at a level of 3 to 7 ppb. Further, hexanal is known to be present in the breath of healthy individuals at a level of 9 to 13 ppb. These or other compounds can be used by the system herein in combination with evaluation of cannabinoid compounds.

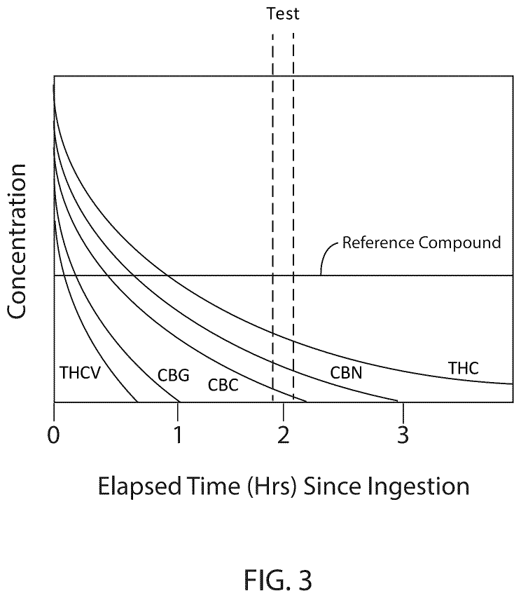

Referring now to FIG. 3, a chart showing elimination curves of various cannabinoids and a reference compound. As with FIG. 1, FIG. 3 shows elimination curves for delta-9-tetrahydrocannabinol (THC), cannabinol (CBN), cannabichromene (CBC), cannabigerol (CBG), and tetrahydrocannabivarin (THCV). FIG. 3 also shows a hypothetical concentration for a reference compound. As can be seen, the concentration of the reference compound remains the same over time and thus can be utilized for baseline and/or normalization purposes.

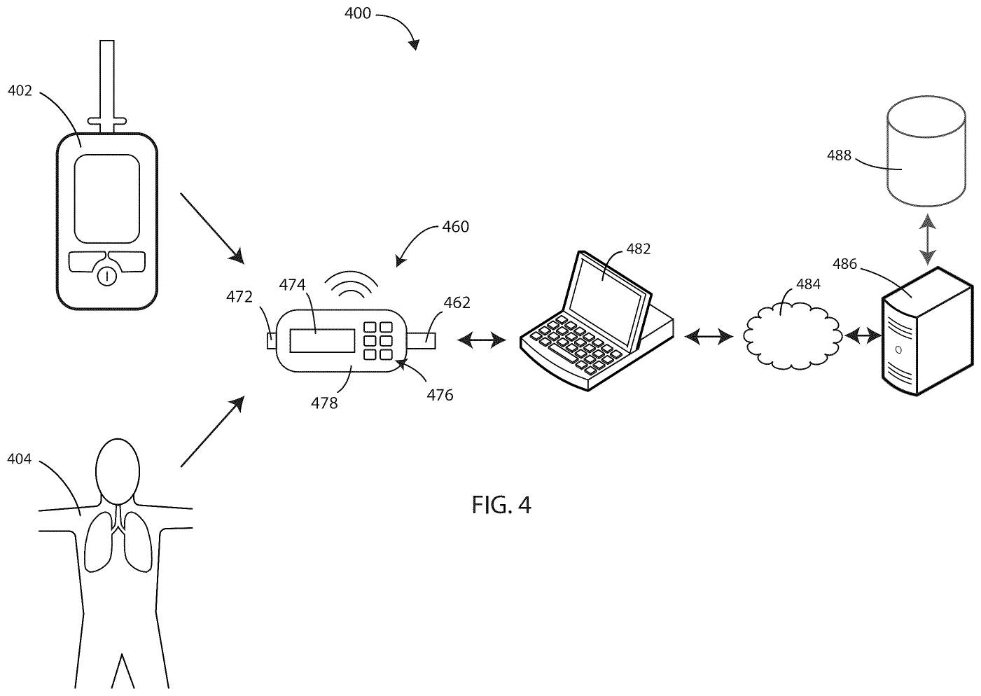

Systems herein can take on various physical forms and include various components. Referring now to FIG. 4, a schematic view of a system 400 for measuring analyte presence in a gaseous sample is shown in accordance with various embodiments herein. The system 400 can include a sensing device 460 that includes a chemical sensor element with a plurality of discrete binding detectors for sensing analytes in a gaseous mixture. The discrete binding detectors can include graphene-based variable capacitors (or graphene varactors), as will be described in more detail below. The terms “discrete binding detector” and “graphene varactor” can be used interchangeably herein unless otherwise specified or the context dictates otherwise.

In the embodiment shown in FIG. 4, the sensing device 460 of system 400 is depicted in a hand-held format that can be used in the field, medical clinic, workplace, and the like. It will be appreciated that the system herein can further include a table-top system for use in a medical clinic, hospital, laboratory, etc. However, it will be appreciated that many other formats for the sensing device 460 and system 400 are also contemplated herein.

The sensing device 460 can include a housing 478 and an air intake port 462. In some embodiments, air intake port 462 can be in fluid communication with one or more separate gas sampling devices 402. In other embodiments, air intake port 462 can be configured as a mouthpiece into which a subject 404 to be evaluated can blow a breath sample. In yet other embodiments, the air intake port 462 can itself act as a gas sampling device. The sensing device 460 can be configured to actively draw a gas into housing 478 or it can be configured to receive a gas passively from a subject 404 or a gas sampling device 402. In some embodiments, the sensing device 460 can include a flow control valve in fluid communication with an upstream flow path relative the chemical sensor element. In various embodiments, the flow control valve can control fluid communication between an upstream flow path and the chemical sensor element.

The sensing device 460 can also include a display screen 474 and a user input device 476, such as a keyboard. The sensing device 460 can also include a gas outflow port 472. In some embodiments, the system 400 can include a local computing device 482 that can include a microprocessor, input and output circuits, input devices, a visual display, a user interface, and the like. In some embodiments, the sensing device 460 can communicate with the local computing device 482 in order to exchange data between the sensing device 460 and the local computing device 482. The local computing device 482 can be configured to perform various processing steps with the data received from the sensing device 460, including, but not limited to, calculating various parameters of the graphene varactors described herein. However, it should be appreciated that in some embodiments the features associated with the local computing device 482 can be integrated into the sensing device 460. In some embodiments, the local computing device 482 can be a laptop computer, a desktop computer, a server (real or virtual), a purpose dedicated computer device, or a portable computing device (including, but not limited to, a mobile phone, tablet, wearable device, etc.). The local computing device 482 and/or the sensing device 460 can communicate with computing devices in remote locations through a data network 484, such as the Internet or another network for the exchange of data as packets, frames, or otherwise.

In some case, various operations can be performed in order to facilitate processing at the edge (e.g., with the sensing device 460 and/or a local computing device 482). By way of example, in some cases template patterns used herein, half-life data, and/or half-life elimination curves for cannabinoids and/or metabolites, or other data can be saved in memory by the sensing device 460 and/or a local computing device 482 for use in executing operations herein. In some embodiments, some data can be discarded or otherwise not used in order to simplify calculations herein. By way of example, in some embodiments, the system can be configured to drop, discard, or otherwise not utilize data relating to compounds other than those of relevance for analyzing cannabinoid compounds and/or metabolites thereof. In some embodiments, the system can be configured to drop, discard, or otherwise not utilize data relating to compounds other than cyclic and/or aromatic compounds.

However, in some embodiments, various operations herein can be performed, at least in part, by remote computing resources. For example, in some embodiments, the system 400 can also include a computing device such as a server 486 (real or virtual). In some embodiments, the server 486 can be located remotely from the sensing device 460. The server 486 can be in data communication with a database 488. The database 488 can be used to store various subject information, such as that described herein. In some embodiments, the database 488 can specifically include characteristic patterns of data (or templates) associated with cannabinoid use at multiple different time points after ingestion (such as 5, 10, 15, 20, 25, 30, 40, 50, 60, 70, 80, 90, 100, 110, 120, 150, 180, 210, 240, 300, 360 minutes after ingestion, or longer, or an amount falling within a range between any of the foregoing), half-lives of cannabinoids, half-lives of cannabinoid metabolites, elimination curves of cannabinoids, elimination curves of cannabinoid metabolites, and the like.

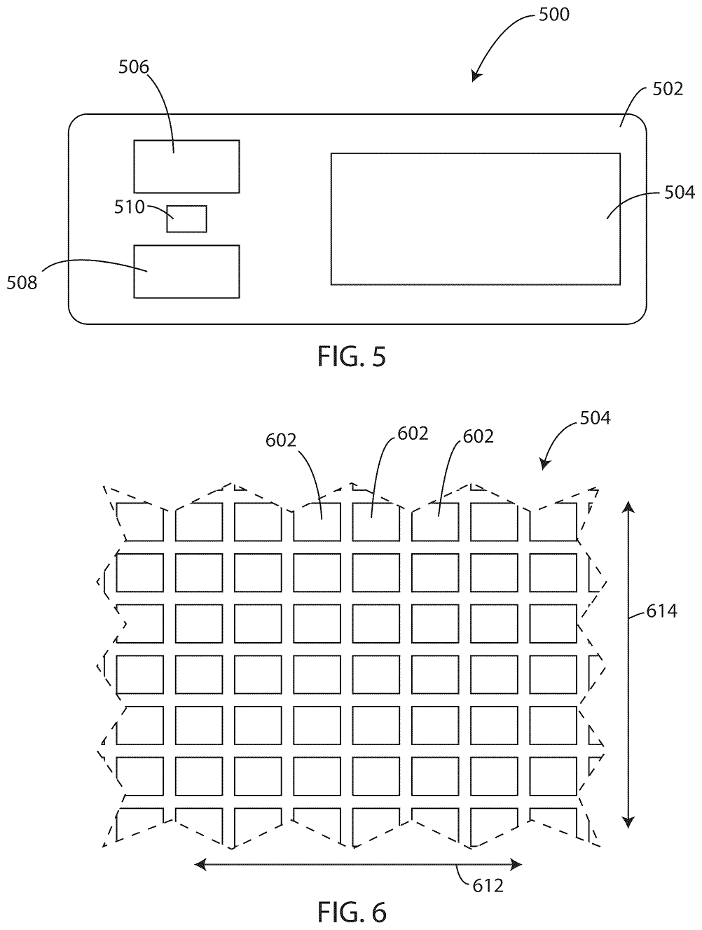

Referring now to FIG. 5, a schematic top plan view of a chemical sensor element 500 is shown in accordance with various embodiments herein. The chemical sensor element 500 can include a substrate 502. It will be appreciated that the substrate can be formed from many different materials, including silicon, glass, quartz, sapphire, polymers, metals, glasses, ceramics, cellulosic materials, composites, metal oxides, and the like. The thickness of the substrate can vary. In some embodiments, the substrate has sufficient structural integrity to be handled without undue flexure that could damage components thereon.

The chemical sensor elements herein can include a first measurement zone 504, and second measurement zone 506, and a third measurement zone 508 that can be disposed on the substrate 502. It will be appreciated that more than three measurement zones can be present on the chemical sensor elements herein. In some embodiments, the first measurement zone 504 can define at least a portion of a first gas flow path. The first measurement zone 504 can include a plurality of discrete binding detectors that can sense analytes in a gaseous sample, such as a breath sample. The second measurement zone 506 can define at least a portion of a second gas flow path. In some embodiments, the second gas flow path can be entirely separate from the first gas flow path, while in other embodiments the second gas flow path can include a portion of the first gas flow path.

The second measurement zone 506 can also include a plurality of discrete binding detectors. The chemical sensor element can include a component 510 to store reference data. The component 510 to store reference data can be an electronic data storage device, an optical data storage device, a printed data storage device (such as a printed code), or the like. The chemical sensor elements described herein can be as described in more detail in U.S. Publ. No. U.S. 2016/0109440 A1, which is herein incorporated by reference in its entirety.

Each chemical sensor element herein can include one or more discrete binding detectors in an array throughout the measurement zones. Referring now to FIG. 6, a schematic diagram of a portion of a chemical sensor element is shown in accordance with various embodiments herein. A plurality of graphene varactors 602 can be disposed on the first measurement zone 504 in an array within the measurement zones. In some embodiments, a chemical sensor element can include a plurality of graphene varactors configured in an array. In some embodiments, the plurality of graphene varactors can include identical surface chemistries, while in other embodiments the plurality of graphene varactors can include different surface chemistries from one another. In some embodiments, graphene varactors having the same surface chemistries can be present in duplicate, triplicate, or more, such that data obtained during measurement cycles can be averaged together to further refine the change observed in the response signals. The graphene varactors herein can be as described in more detail in U.S. Pat. No. 9,513,244, which is herein incorporated by reference in its entirety. It will be appreciated that any of the first measurement zone, the second measurement zone, the third measurement zone, and the like can include one or more arrays of a plurality of graphene varactors as described herein.

In some embodiments, the graphene varactors can be heterogeneous in that they are different (in groups or as individual graphene varactors) from one another in terms of their binding behavior or specificity with regard a particular analyte. In some embodiments, some graphene varactors can be duplicated, triplicated, or more, for validation purposes but are otherwise heterogeneous from other graphene varactors. Yet in other embodiments, the graphene varactors can be homogeneous. While the graphene varactors 602 of FIG. 6 are shown as boxes organized into a grid, it will be appreciated that the graphene varactors can take on many different shapes (including, but not limited to, various polygons, circles, ovals, irregular shapes, and the like) and, in turn, the groups of graphene varactors can be arranged into many different patterns (including, but not limited to, star patterns, zig-zag patterns, radial patterns, symbolic patterns, and the like).

In some embodiments, the order of specific graphene varactors 602 across a length 612 and width 614 of the measurement zone can be substantially random. In other embodiments, the order can be specific. For example, in some embodiments, a measurement zone can be ordered so that the specific graphene varactors 602 configured to bind to analytes having a lower molecular weight are located farther away from the incoming gas flow relative to specific graphene varactors 602 configured to bind to analytes having a higher molecular weight which are located closer to the incoming gas flow. As such, chromatographic effects which may serve to provide separation between chemical compounds of different molecular weight can be taken advantage of to provide for optimal binding of chemical compounds to corresponding graphene varactors.

The number of graphene varactors can be from about 1 to about 100,000. In some embodiments, the number of graphene varactors can be from about 1 to about 10,000. In some embodiments, the number of graphene varactors can be from about 1 to about 1,000. In some embodiments, the number of graphene varactors can be from about 2 to about 500. In some embodiments, the number of graphene varactors can be from about 10 to about 500. In some embodiments, the number of graphene varactors can be from about 50 to about 500. In some embodiments, the number of graphene varactors can be from about 1 to about 250. In some embodiments, the number of graphene varactors can be from about 1 to about 50.

The graphene varactor or other graphene sensor herein can include a graphene layer and a self-assembled monolayer disposed on an outer surface of the graphene layer through π-π stacking interactions. Graphene is a form of carbon containing a single layer of carbon atoms in a hexagonal lattice. Graphene has a high strength and stability due to its tightly packed sp2 hybridized orbitals, where each carbon atom forms one sigma (σ) bond each with its three neighboring carbon atoms and has one p orbital projected out of the hexagonal plane. The p orbitals of the hexagonal lattice can hybridize to form a π bond suitable for non-covalent π-π stacking interactions with other π-electron rich molecules.

The non-covalent functionalization of graphene with a self-assembled monolayer does not significantly affect the atomic structure of graphene, and provides a stable graphene-based sensor with high sensitivity towards a number of volatile organic compounds (VOCs) in the parts-per-billion (ppb) or parts-per-million (ppm) levels. The use of specific compounds with which to form a self-assembled monolayer on the graphene can increase the sensitivity towards cannabinoid compounds and/or metabolites thereof allowing for collection of a richer dataset to more accurately determine a time window of cannabinoid use.

As such, the graphene varactors described herein can include those in which a single graphene layer has been surface-modified through non-covalent π-π stacking interactions between graphene and π-electron-rich molecules, such as, for example, at least one of pyrenes, coronenes, aromatic cyclodextrins, and pillarenes. In some embodiments, at least two of pyrenes, coronenes, aromatic cyclodextrins, and pillarenes are included on graphene surfaces herein. In some embodiments, at least three of pyrenes, coronenes, aromatic cyclodextrins, and pillarenes are included on graphene surfaces herein. In some embodiments, at least four of pyrenes, coronenes, aromatic cyclodextrins, and pillarenes are included on graphene surfaces herein. Aromatic cyclodextrins can include benzylated cyclodextrins, such as α-, β- and γ-cyclodextrins derivatives, including, but not limited to, α-CDBn18, β-CDBn21, γ-CDBn24, β-CDBn19(OH)2.

In some embodiments, the self-assembled monolayer can provide at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% surface coverage (by area) of the graphene layer. It will be appreciated that the self-assembled monolayer can provide surface coverage falling within a range wherein any of the forgoing percentages can serve as the lower or upper bound of the range, provided that the lower bound of the range is a value less than the upper bound of the range.

In some embodiments, it will be appreciated that the self-assembly of π-electron-rich molecules on the surface of the graphene layer can include the self-assembly into more than a monolayer, such as a multilayer. Multilayers can be detected and quantified by techniques such as scanning tunneling microscopy (STM) and other scanning probe microscopies. References herein to a percentage of coverage greater than 100% shall refer to the circumstance where a portion of the surface area is covered by more than a monolayer, such as covered by two, three or potentially more layers of the compound used. Thus, a reference to 105% coverage herein shall indicate that approximately 5% of the surface area includes more than monolayer coverage over the graphene layer. In some embodiments, graphene surfaces can include 101%, 102%, 103%, 104%, 105%, 110%, 120%, 130%, 140%, 150%, or 175% surface coverage of the graphene layer. It will be appreciated that multilayer surface coverage of the graphene layer can fall within a range of surface coverages, wherein any of the forgoing percentages can serve as the lower or upper bound of the range, provided that the lower bound of the range is a value less than the upper bound of the range. For example, ranges of coverage can include, but are not limited to, 50% to 150% by surface area, 80% to 120% by surface area, 90% to 110%, or 99% to 120% by surface area.

In some embodiments, the self-assembled monolayers suitable for use herein can provide coverage of the graphene surface with a monolayer as quantified by the Langmuir theta value of at least some minimum threshold value, but avoid covering the majority of the surface of the graphene with a multilayer thicker than a monolayer. In some embodiments, the self-assembled monolayers suitable for use herein provide a Langmuir theta value of at least 0.95. In some embodiments, the self-assembled monolayers suitable for use herein provide a Langmuir theta value of at least 0.98. In some embodiments, the self-assembled monolayers can provide a Langmuir theta value of at least 0.85, 0.86, 0.87, 0.88, 0.89, 0.90, 0.91, 0.92, 0.93, 0.94, 0.95, 0.96, 0.97, 0.98, 0.99, or 1.0. It will be appreciated that the self-assembled monolayer can provide a range of Langmuir theta values, wherein any of the forgoing Langmuir theta values can serve as the lower or upper bound of the range, provided that the lower bound of the range is a value less than the upper bound of the range.

In some embodiments, each of the graphene varactors suitable for use herein can include at least a portion of one or more electrical circuits. By way of example, in some embodiments, each of the graphene varactors can include all or a portion of one or more passive electrical circuits or active electrical circuits. In some embodiments, the graphene varactors herein can include two-terminal graphene varactors. In some embodiments, the two-terminal graphene varactors can be adapted to each receive independent signals from an electrical signal generator. In some embodiments, the graphene varactors can be formed such that they are integrated directly on an electronic circuit. In some embodiments, the graphene varactors can be formed such that they are wafer bonded to the circuit. In some embodiments, the graphene varactors can include integrated readout electronics, such as a readout integrated circuit (ROIC). The electrical properties of the electrical circuit, including resistance or capacitance, can change upon binding, such as specific and/or non-specific binding, with a compound from a biological sample. Many different types of circuits can be used to gather data from chemical sensor elements and will be discussed below in reference to FIGS. 9 and 10.

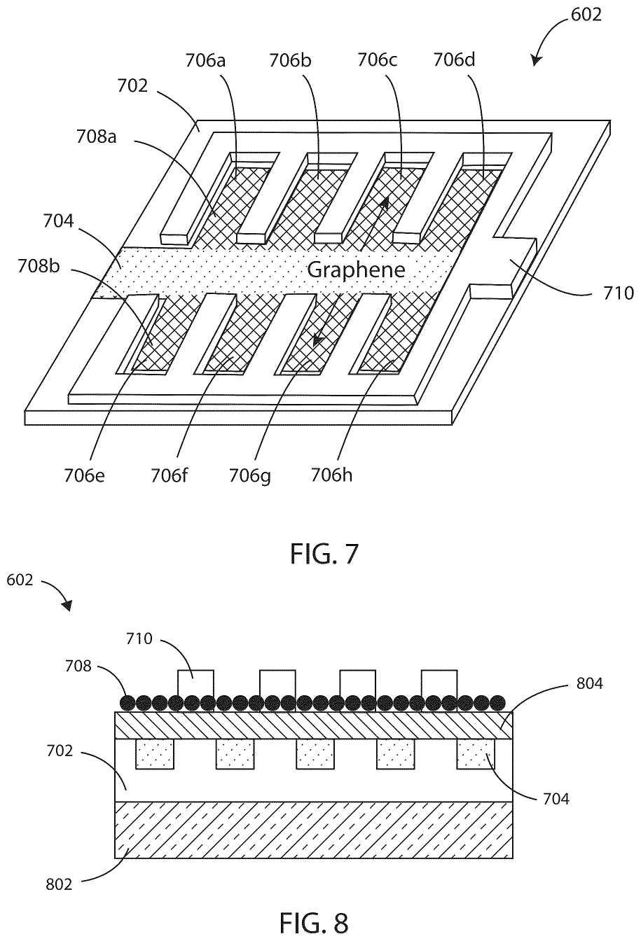

Referring now to FIG. 7, a schematic view of a graphene varactor 602 having two terminals is shown in accordance with the embodiments herein. It will be appreciated that graphene varactors can be prepared in various ways with various geometries, and that the graphene varactor shown in FIG. 7 is just one example in accordance with the embodiments herein.

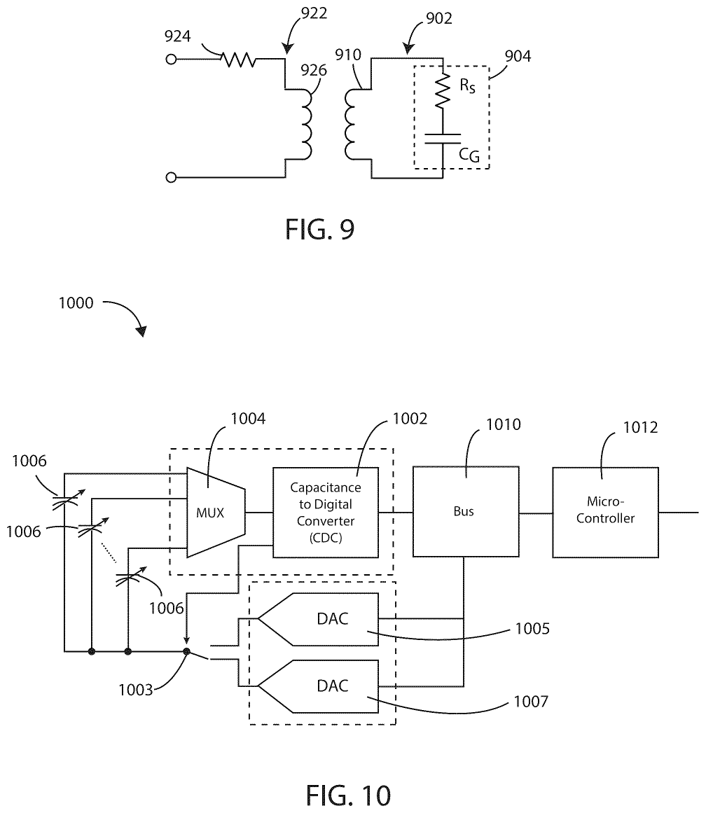

Graphene varactor 602 can include an insulator layer 702, a gate electrode 704 (or “gate contact”), a dielectric layer (item 804 in FIG. 8), one or more graphene layers, such as graphene layers 708a and 708b, and a contact electrode 710 (or “graphene contact”). In some embodiments, the graphene layer(s) 708a-b can be contiguous, while in other embodiments the graphene layer(s) 708a-b can be non-contiguous. Gate electrode 704 can be deposited within one or more depressions formed in insulator layer 702. Insulator layer 702 can be formed from an insulative material such as silicon dioxide, formed on a substrate (item 802 in FIG. 8), such as a silicon substrate (wafer), and the like. Gate electrode 704 can be formed by an electrically conductive material such as chromium, copper, gold, silver, tungsten, aluminum, titanium, palladium, platinum, iridium, and any combinations or alloys thereof, which can be deposited on top of or embedded within the insulator layer 702. The dielectric layer (not shown in FIG. 7) can be disposed on a surface of the insulator layer 702 and the gate electrode 704, as shown in more detail in FIG. 8. The graphene layer(s) 708a-b can be disposed on the dielectric layer.

Graphene varactor 602 includes eight gate electrode fingers 706a-706h. It will be appreciated that while graphene varactor 602 shows eight gate electrode fingers 706a-706h, any number of gate electrode finger configurations can be contemplated. In some embodiments, an individual graphene varactor can include fewer than eight gate electrode fingers. In some embodiments, an individual graphene varactor can include more than eight gate electrode fingers. In other embodiments, an individual graphene varactor can include two gate electrode fingers. In some embodiments, an individual graphene varactor can include 1, 2, 3, 4, 5, 6, 7, 8, 9, 10, or more gate electrode fingers.

Graphene varactor 602 can include one or more contact electrodes 710 disposed on portions of the graphene layers 708a and 708b. Contact electrode 710 can be formed from an electrically conductive material such as chromium, copper, gold, silver, tungsten, aluminum, titanium, palladium, platinum, iridium, and any combinations or alloys thereof. Further aspects of exemplary graphene varactors can be found in U.S. Pat. No. 9,513,244, the content of which is herein incorporated by reference in its entirety.

Referring now to FIG. 8, a schematic diagram of a portion of a cross-sectional view of an exemplary graphene varactor is shown in accordance with various embodiments herein. The graphene varactor can include a substrate 802, such as a silicon substrate (wafer). An insulator layer 702 can be disposed on the substrate 802, and a gate electrode 704 can be recessed into the insulator layer 702. The gate electrode 704 can be formed by depositing an electrically conductive material in the depression in the insulator layer 702, as discussed above in reference to FIG. 7. A dielectric layer 804 can be formed on a surface of the insulator layer 702 and the gate electrode 704. In some examples, the dielectric layer 804 can be formed of a material, such as, silicon dioxide, aluminum oxide, hafnium dioxide, zirconium dioxide, hafnium silicate, or zirconium silicate. The graphene layer 708 is disposed on the dielectric layer 804 and the contact electrode 710 can be disposed in contact with the graphene layer 708. In some examples, the dielectric layer 804 can include multiple layers of the dielectric materials listed herein. In some embodiments, the dielectric layer 804 can include alternating layers of different dielectric materials. In some embodiments, the dielectric layer 804 can include alternating layers of aluminum oxide and hafnium dioxide.

In some embodiments herein, to maintain the stability of the graphene varactors herein, the chemical sensor elements can be pretreated under a vacuum at a temperature from 50° C. to 150° C. for at least 3 hours. In various embodiments, the chemical sensor elements can be pretreated under vacuum at a temperature from 120° C. to 150° C. for 10 to 20 hours. In various embodiments, the chemical sensor elements can be pretreated under a vacuum at a temperature can be greater than or equal to 50° C., 60° C., 70° C., 80° C., 90° C., 100° C., 110° C., 120° C., 130° C., 140° C., 150° C., 160° C., 170° C., 180° C., 190° C., or 200° C., or can be an amount falling within a range between any of the foregoing.

In addition, the chemical sensor elements herein can be maintained under a controlled gas environment until it is exposed to a gaseous test sample. By way of example, the chemical sensor element can be maintained under a controlled gas environment including oxygen gas, nitrogen gas or an inert gas such as, for example, argon, helium, xenon, krypton, or neon.

In many cases, each graphene varactor can be interrogated using a number of different applied voltages (a plurality of voltages) with the resulting data forming a C-V_{g} curve. The plurality of voltages can fall within a range from a lower voltage bound to an upper voltage bound. In many cases the voltages may start at the lower bound and then be increased progressing up to the upper bound, thus sweeping across the range in a first direction followed by a sweep in the opposite (or second) direction (e.g., from the upper bound to the lower bound). Thus, in various embodiments, a first direction can include a sweep from the lower voltage bound to the upper voltage bound and a second direction is a sweep from the upper voltage bound to the lower voltage bound. However, in other embodiments, the first direction can include a sweep from the upper voltage bound to the lower voltage bound and the second direction is a sweep from the lower voltage bound to the upper voltage bound. In various embodiments, a sweep in the first direction followed by a sweep in the second direction constitutes a measurement cycle.

The values for the lower voltage bound and the upper voltage bounds can be predetermined or can be determined dynamically. In various embodiments, the lower voltage bound and the upper voltage bound are preset values and can be selected from values such as −3.0 V or less, −2.9 V, −2.8 V, −2.7 V, −2.6 V, −2.5 V, −2.4 V, −2.3 V, −2.2 V, −2.1 V, −2.0 V, −1.9 V, −1.8 V, −1.7 V, −1.6 V, −1.5 V, −1.4 V, −1.3 V, −1.2 V, −1.1 V, −1.0 V, −0.9 V, −0.8 V, −0.7 V, −0.6 V, −0.5 V, −0.4 V, −0.3 V, −0.2 V, −0.1 V, 0 V, 0.1 V, 0.2 V, 0.3 V, 0.4 V, 0.5 V, 0.6 V, 0.7 V, 0.8 V, 0.9 V, 1.0 V, 1.1 V, 1.2 V, 1.3 V, 1.4 V, 1.5 V, 1.6 V, 1.7 V, 1.8 V, 1.9 V, 2.0 V, 2.1 V, 2.2 V, 2.3 V, 2.4 V. 2.5 V, 2.6 V, 2.7 V, 2.8 V, 2.9 V, or 3.0 V or more, or a voltage value falling between any of the foregoing values. In various embodiments, the lower voltage bound and the upper voltage bound are preset values and can be selected from values ranging from −5 V to 5 V; from −4 V to 4 V; from −3 V to 3 V; from −2 V to 2 V; from −1.5 V to 1.5V; or from −1 V to 1 V.

While an instantaneous applied voltage herein can be thought of as the sum of a DC bias component and an AC component, it will be appreciated that specific applied voltages values as referenced herein typically represent the DC voltage bias or offset value. This is because the average value of an AC component over a non-instantaneous time period will be zero. As such, unless otherwise stated to the contrary or the context dictates otherwise, voltage value references herein shall refer to the DC bias or offset component of an applied voltage, understanding that corresponding instantaneous voltage values can vary based on the AC component. The waveforms of the AC component can take many different forms. For example, they can be sinusoidal, square, triangular, trapezoidal, ramped, sawtooth, complex, or the like.

In some embodiments, the lower voltage bound and the upper voltage bound are dynamically determined values. For example, the bounds can be changed based on previously applied excitation voltages and/or previously observed values related to the graphene sensor and/or previously observed effects.

In some embodiments the upper voltage bound and the lower voltage bound is static between successive measurement cycles. In other embodiments, the upper voltage bound and the lower voltage bound may change between successive measurement cycles. For example, in some embodiments, the first measurement cycle can include the use of the widest range of excitation voltages and successive measurement cycles may utilize a narrower range of excitation voltages.

Various timing schemes can be used for the sweep across a range of voltages. In some embodiments, a sweep in the first direction can be immediately followed by a sweep in the second direction. In other embodiments, a sweep in the first direction can be followed by a pause and then a sweep in the second direction. The duration of a pause between sweeps can include those from 1 millisecond (ms) to 5 seconds in length. In some embodiments, the duration of the pause between sweeps can be greater than or equal to, 10 ms, 20 ms, 30 ms, 40 ms, 50 ms, 60 ms, 70 ms, 80 ms, 90 ms, 100 ms, 200 ms, 300 ms, 400 ms, 500 ms, 600 ms, 700 ms, 800 ms, 900 ms, or 1 sec, 2 sec, 3 sec, 4 sec, or 5 sec, or can be an amount falling within a range between any of the foregoing.

In some embodiments, the duration of a pause between sweeps can be greater than 5 seconds in length. In various embodiments, the duration of a pause between sweeps can greater than or equal to 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, or 60 sec, or can be an amount falling within a range between any of the foregoing. In other embodiments, the duration of a pause between sweeps can be greater than 1 minute.

A change in any one of the parameters of the capacitance versus voltage values provides data that can reflect the binding status of analytes to the graphene varactor(s), and can be used to characterize a sample and/or distinguish various analytes and analyte concentrations in the sample.

Various measurable aspects can be used to characterize the content of a sample (such as a breath sample) herein to determine cannabinoid content and serve as data for use in estimating a time of cannabinoid ingestion. In some embodiments, a ratio of the maximum capacitance to minimum capacitance can be used to characterize the content of a gaseous mixture. In some embodiments, a ratio of the maximum capacitance to the shift in the Dirac point can be used to characterize the content of a gaseous mixture. In other embodiments, a ratio of the minimum capacitance to the shift in the slope of the response signal can be used to characterize the content of a gaseous mixture. In some embodiments, a ratio of any of the parameters including a shift in the Dirac point, a change in the minimum capacitance, a change in the slope of the response signal, or the change in the maximum capacitance can be used to characterize the content of a sample mixture. In accordance with embodiments herein, hysteresis effects observed with respect to any of these values (as well as other types of values discussed) can be used to characterize the content of sample mixtures.

Various measurement circuitry can be used to measure the changes in the parameters of the capacitance-voltage curve of the graphene varactor(s). Measurement circuitry suitable for use herein can include active and passive sensing circuits. Such circuitry can implement wired (direct electrical contact) or wireless sensing techniques. Referring now to FIG. 9, a schematic diagram of a passive sensor circuit 902 and a portion of a reading circuit 922 is shown in accordance with various aspects herein. In some embodiments, the passive sensor circuit 902 can include a metal-oxide-graphene varactor 904 (wherein RS represents the series resistance and CG represents the varactor capacitor) coupled to an inductor 910. In some embodiments, the reading circuit 922 can include a reading coil having a resistance 924 and an inductance 926.

Measurement circuitry herein can also include active sensing circuits. In various embodiments, the measurement circuity can include an electrical signal generator configured to generate a series of measurement cycles over a time period. The measurement circuity can include an electrical signal generator configured to generate and deliver an applied voltage that can be represented as an alternating voltage (or excitation voltage) superimposed on a bias voltage. It will be appreciated that there are many ways to generate such an applied voltage.

In some embodiments, measurement circuity can include an electrical signal generator configured to generate and deliver an applied voltage that includes a sinusoidal, square, triangular, trapezoidal, ramped, sawtooth, or complex waveform alternating voltage superimposed on a bias voltage. In some embodiments, the electrical signal generator can be configured to generate an applied voltage at a plurality of voltages to be applied to the one or more graphene varactors, the voltages falling within a range from a lower voltage bound and an upper voltage bound, the voltages starting at one bound and moving to the other bound as part of a sweep across the voltages. In some embodiments, the electrical signal generator can be configured to generate an excitation current at a plurality of voltages to be applied to the one or more graphene varactors, the voltages falling within a range from a lower bound and an upper bound, the voltages starting at one bound and moving to the other bound as part of a sweep across the voltages.

Referring now to FIG. 10, a schematic diagram is shown of an example of measurement circuity 1000 to measure the capacitance of a plurality of graphene sensors in accordance with various embodiments herein. The measurement circuity 1000 can include a capacitance to digital converter (CDC) 1002 in electrical communication with a multiplexor 1004. The multiplexor 1004 can provide selective electrical communication with a plurality of graphene varactors 1006. The connection to the other side of the graphene varactors 1006 can be controlled by a switch 1003 (as controlled by the CDC) and can provide selective electrical communication with a first digital to analog converter (DAC) 1005 and a second digital to analog converter (DAC) 1007. The other side of the DACs 1005, 1007 can be connected to a bus device 1010, or in some cases, the CDC 1002. The circuitry can further include a microcontroller 1012 (or controller circuit), which will be discussed in more detail below.

In this case, a signal from the CDC controls the switch 1003 between the output voltages of the two programmable Digital to Analog Converters (DACs) 1005 and 1007. The programmed voltage difference between the DACs determines an excitation amplitude (and represents the AC component of the applied voltage), providing an additional programmable scale factor to the measurement and allowing measurement of a wider range of capacitances than specified by the CDC. The bias voltage at which the capacitance is measured is equal to the difference between the bias voltage at the CDC input (via the multiplexor, usually equal to VCC/2, where VCC is the supply voltage) and the average voltage of the excitation signal, which is programmable. In some embodiments, buffer amplifiers and/or bypass capacitance can be used at the DAC outputs to maintain stable voltages during switching. It will be appreciated that the circuits of FIGS. 9 and 10 are merely exemplary. Many different approaches are contemplated herein.

The measurement circuity can include a capacitance sensor configured to measure capacitance of the discrete binding detectors resulting from the excitation voltage. The measurement circuity can also include a controller circuit configured to determine a change in at least one of a measured capacitance versus voltage value and a calculated value based on the measured capacitance or voltage over the time period. In various embodiments, the measured capacitance versus voltage values can include one or more of a capacitance at a particular voltage, a maximum slope of capacitance to voltage, a minimum slope of capacitance to voltage, a minimum capacitance, a voltage at minimum capacitance (Dirac voltage), a maximum capacitance, and a ratio of maximum capacitance to minimum capacitance. In various embodiments, the controller circuit is configured to measure a difference between a forward Dirac point voltage and a reverse Dirac point voltage. In some embodiments, the controller circuit is configured to calculate a rate of change of measured capacitance over the time period at multiple discrete DC bias voltages. In some embodiments, the controller circuit is configured to calculate an average hysteresis change value of a measured property over a plurality of measurement cycles. In various embodiments, the controller circuit is configured to determine the forward Dirac point voltage and/or the reverse Dirac point voltage.

In some embodiments, the measurement circuitry or another part of the system herein can include a temperature controller configured to control a temperature of the graphene varactors. In some embodiments, the temperature controller can include a thermistor, thermocouple, resistive thermal device (RTD) and the like. In various embodiments, controlling the temperature of the graphene varactors comprises exposing the graphene varactor to one or more temperature set points for a predetermined time. In some embodiments, a sequence involving increasing the temperature set points over a course of a predetermined time can be used. In other embodiments, a sequence involving decreasing the temperature set points over a course of a predetermined time can be used. In other embodiments, a sequence involving increasing the temperature set points followed by decreasing the temperature set points can be used.

The system for measuring analyte presence in a gaseous sample can be configured to measure differences in a capacitance versus voltage value when an applied voltage is swept in a first direction between the lower voltage bound and upper voltage bound versus a second direction between the between the upper voltage bound and lower voltage bound. In various embodiments, the first direction is a sweep from the lower voltage bound to the upper voltage bound and the second direction is a sweep from the upper voltage bound to the lower voltage bound. In various embodiments, the first direction is a sweep from the upper voltage bound to the lower voltage bound and the second direction is a sweep from the lower voltage bound to the upper voltage bound.

Various values for the voltages suitable for use within a range from a lower bound to an upper bound as contemplated herein are described further below. In various embodiments, each measurement cycle includes delivering a DC bias voltage to the discrete binding detectors at multiple discrete DC bias voltage values across a range of DC bias voltages as discussed in greater detail below.

In some cases, the above calculated values can be indicative of the identity and/or concentrations of specific volatile organic components of a gas sample, such as specific cannabinoids. As such, each of the calculated values above can serve as a distinct piece of data that forms part of a pattern for a given subject and/or given gas sample. As also described elsewhere herein, the pattern can then be matched against preexisting patterns, or patterns identified in real-time, derived from large, stored data sets through techniques such as machine learning or other techniques, wherein such patterns are determined to be characteristic of cannabinoid use at specific times post-ingestion. The above calculated aspects can also be put to other purposes, diagnostic and otherwise.

In some embodiments, calculations such as those described above can be performed by a controller circuit. The controller circuit can be configured to receive an electrical signal reflecting the capacitance or voltage of the graphene varactors. In some embodiments, the controller circuit can include a microcontroller to perform these calculations. In some embodiments, the controller circuit can include a microprocessor in electrical communication with the measurement circuity. The microprocessor system can include components such as an address bus, a data bus, a control bus, a clock, a CPU, a processing device, an address decoder, RAM, ROM and the like. In some embodiments, the controller circuit can include a calculation circuit (such as an application specific integrated circuit—ASIC) in electrical communication with the measurement circuity.

In addition, in some embodiments, the system can include a nonvolatile memory. In some embodiments, the non-volatile memory can be configured to store measured capacitance values for the discrete binding detectors across a range of DC bias voltages. In other embodiments, the nonvolatile memory can be configured to store a baseline capacitance for the discrete binding detectors across a range of DC bias voltages. In some embodiments, the nonvolatile memory can be where sensitivity calibration information for the graphene varactors is stored.

By way of example, the graphene varactors can be tested in a production facility, where sensitivity to various analytes such as VOC's can be determined and then stored on an EPROM or similar component. In addition, or alternatively, sensitivity calibration information can be stored in a central database and referenced with a chemical sensor element serial number when subject data is sent to a central location for analysis and diagnosis. These components can be included with any of the pieces of hardware described herein.

In some embodiments herein, components can be configured to communicate over a network, such as the internet or a similar network. In various embodiments, a central storage and data processing facility can be included. In some embodiments, data gathered from sensors in the presence of the subject (local) can be sent to the central processing facility (remote) via the internet or a similar network, and the pattern from the particular subject being evaluated can be compared against those reflecting cannabinoid profiles or patterns at various time points post-ingestion.

### Pattern Matching

Pattern matching algorithms can be used to match the current subject's pattern against predetermined patterns that correlate with (and can therefore indicate) specific amounts of time post-ingestion of a cannabinoid containing substance. Thus, in various embodiments herein, the system can compare a data set reflecting a particular patient/individual against one or more previously determined patterns using a pattern matching or pattern recognition algorithm to determine the pattern that is the best match, wherein the specific previously determined pattern that is the best match indicates the amount of time that has passed since cannabinoid ingestion.

By way of example, pre-determined patterns reflecting amounts of time since cannabinoid use can be identified through machine learning analysis or another similar algorithmic technique by evaluating large sets of data wherein the amount of time that has passed since cannabinoid use is known (a training set of data) so as to facilitate a supervised machine learning approach. Such a training set of data can be processed with a machine learning algorithm or similar algorithmic technique in order to generate one or more patterns of data reflecting the amount of time that has passed since cannabinoid use.

Algorithms can be used herein to create new patterns/models using any of numerous machine learning techniques, or apply the results of previously calculated models using these techniques, such as logistic regression, random forest, or an artificial neural network. Many different pattern matching or pattern recognition algorithms can be used. By way of example, in some embodiments a least squares algorithm can be used to identify a particular pre-determined pattern that a combined data set most closely matches. In various embodiments, standard pattern classification methods can be used including, but not limited to, Gaussian mixture models, clustering, hidden Markov models, as well as Bayesian approaches, neural network models, and deep learning.

After a pattern matching operation herein, an amount of time since cannabinoid use can be estimated. In some embodiments this can be performed remotely and provided back across the data network to the facility where the subject is currently located. In other embodiments, such operations can be performed locally on a device of the system.

### Applied Voltages/Measurement Cycles

A measurement cycle herein can include a sweep through voltages in a first direction followed by a sweep through voltages in the opposite direction to observe measurable parameters and/or changes in measurable parameters. Many different ranges of applied voltages can be used for each measurement cycle. In some embodiments, the applied voltages used in the methods herein can include from −6.0 V, −5.0 V, −4.0 V, −3.0 V, −2.5 V, −2.0 V, −1.5 V, −1.0 V, −0.5 V, 0.5 V, 1.0 V, 1.5 V, 2.0 V, 2.5 V, 3.0 V, 4.0 V, 5.0 V, and 6.0V. It will be appreciated that the applied voltages used in the methods herein can include delivering an applied voltage within a range, wherein any of the forgoing voltages can serve as the lower or upper bound of the range, provided that the lower bound of the range is a value less than the upper bound of the range.

In various embodiments, a “sweep” across a voltage range can include a number of discrete measurements being made during the sweep at a number of discrete bias voltages across the voltage range. In some embodiments, a measurement cycle herein can include a forward sweep (from low applied voltages to high applied voltages). In some embodiments, a measurement cycle herein can include a backward sweep (from high applied voltages to low applied voltages). In some embodiments, a measurement cycle herein can include both a forward and backward sweep. In some embodiments, a measurement cycle herein can include both a forward and backward sweep or any combination thereof.

In some embodiments, a voltage of 0 V or 0.5 V (or other “reset” voltage) can be applied at the end of a measurement cycle and before the next measurement cycle or at the end of all testing.

The length of time for each measurement cycle can depend on various factors including the total number of measurements made of capacitance during the cycle, the total bias voltage range being covered, the voltage step size for each measurement, the time for each measurement, etc. In some embodiments, the time period for each measurement cycle can be about 0.1, 0.5, 1, 2, 3, 4, 5, 6, 7, 8, 9, 10, 12, 15, 20, 30, 45, 60, 120 seconds or more. It will be appreciated that the time period for each measurement cycle can include a range, wherein any of the forgoing time points can serve as the lower or upper bound of the range, provided that the lower bound of the range is a value less than the upper bound of the range.

The excitation signal can be applied to the graphene varactors at a frequency as dictated by the CDC or other hardware component. The frequency of the applied excitation signal can include a frequency that can be greater than or equal to 10 kHz, 20 kHz, 30 kHz, 40 kHz, 50 kHz, 60 kHz, 70 kHz, 80 kHz, 90 kHz, or 100 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, or 1000 MHz or can be an amount falling within a range, wherein any of the foregoing frequencies can serve as the lower or upper bound of the range, provided that the lower bound of the range is a value less than the upper bound of the range.

In some embodiments, the total time for all measurement cycles can be configured to match the total amount of time for testing of a gaseous sample. In some embodiments, the total time for all measurement cycles can be configured to be equal to a predetermined time that covers a period of interest. In some embodiments, the total time for all measurement cycles can be configured to be equal or greater than the total amount of time for a non-steady state phase (or kinetic phase). In some embodiments, the controller circuit can be configured to determine the start of a non-steady state response phase from each of the discrete binding detectors by assessing a rate of change of measured capacitance over time and initiate measurement cycles at that point. In some embodiments, the controller circuit can be configured to initiate measurement cycles when a signal is received indicating the start of a particular test of gaseous sample, such as receiving a sign from a flow sensor that a sample gas is starting to flow to the discrete binding detectors. In some embodiments, the controller circuit can be configured to determine the end of a non-steady state phase by assessing a rate of change of measured capacitance over time and terminating measurement cycles at that point or reducing the frequency of measurement cycles at that point.

In various embodiments, the total time period for generating a series of measurement cycles (the total time for all measurement cycles) can include from 10 seconds to 1200 seconds. In some embodiments, the time period for generating a series of measurement cycles can include from 30 seconds to 180 seconds. In some embodiments, the time period for generating a series of measurement cycles can include from 10, 15, 20, 25, 30, 40, 45, 60, 90, 120, 150, 180, 360, 540, 720, 1080, 1200 seconds or more. It will be appreciated that the time period for generating a series of measurement cycles can include a range, wherein any of the forgoing time points can serve as the lower or upper bound of the range, provided that the lower bound of the range is a value less than the upper bound of the range.

In some embodiments, stepping through the range of applied voltages can include stepping through the range of applied voltages in predetermined increments, such as 50 mV increments. In some embodiments, stepping through the range of applied voltages can include stepping through the range of applied voltages in 10 mV increments. Stepping through the range of applied voltages can be performed at voltage increments of 1 mV, 5 mV, 10 mV, 25 mV, 50 mV, 75 mV, 100 mV, 125 mV, 150 mV, 200 mV, 300 mV, 400 mV, or 500 mV, or by a stepped amount falling within a range between any of the foregoing. In various embodiments, stepping through the range of applied voltages can include stepping through the range of applied voltages in increments from 1 mV to 500 mV. In various embodiments, stepping through the range of applied voltages can include stepping through the range of applied voltages in increments from 5 mV to 300 mV.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.
In an embodiment, a method of detecting cannabinoid ingestion timing is included, the method including taking measurements using a graphene sensor at a first time and at a second time, and comparing a pattern of the first time with a pattern of the second time and determine a time window of cannabinoid use based on the comparison.
In an embodiment, the method can further include providing a voltage stimulus to the graphene sensor and measuring resulting capacitance values of the graphene sensor elements. In an embodiment, the method can further include estimating cannabinoid and/or cannabinoid metabolite amounts.
In an embodiment, a method of detecting cannabinoid ingestion timing is included, the method taking measurements using a graphene sensor, matching a pattern based on the measurements against a set of predetermined patterns, and determining a time window of cannabinoid use based on a match found amongst the set of predetermined patterns.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A system for detecting cannabinoids in breath comprising:
a sensor device; and
a measurement circuit;
wherein the system is configured to
take measurements at a first time and at a second time; and
compare data of the first time with data of the second time and determine a time window of cannabinoid use based on the comparison.

2. The system of claim 1, wherein the system is configured to compare a data pattern of the first time with a data pattern of the second time and determine a time window of cannabinoid use based on the comparison.

3. The system of claim 1, wherein the measurement circuit is configured to provide a voltage stimulus to the sensor device and measure resulting capacitance values.

4. The system of claim 3, wherein the voltage stimulus is provided over a range of voltage values.

5. The system of claim 1, the sensor device comprising:
a first set of graphene sensor elements; and
a second set of graphene sensor elements.

6. The system of claim 5,
the first set of graphene sensor elements comprising graphene varactors; and
the second set of graphene sensor elements comprising graphene varactors.

7. The system of claim 1, the sensor device comprising a graphene element, wherein a compound is disposed on a surface of the graphene element.

8. The system of claim 7, the compound comprising at least one selected from the group consisting of pyrenes, coronenes, aromatic cyclodextrins, and pillarenes.

9. The system of claim 7, wherein the compound is in the form of a self-assembling monolayer.

10. The system of claim 1, wherein the data reflect amounts of cannabinoid and/or cannabinoid metabolites.

11. The system of claim 1, wherein the data reflect relative amounts of cannabinoid and/or cannabinoid metabolites.

12. The system of claim 1, wherein the data reflect amounts of cannabinoids and/or cannabinoid metabolites with different half-lives in vivo.

13. The system of claim 12, the cannabinoids and/or cannabinoid metabolites comprising at least one selected from the group consisting of delta-9-tetrahydrocannabinol (THC), cannabidiol (CBD), 11-OH-THC, 11-COOH-THC, 7-OH-CBD, and 7-COOH-CBD.

14. The system of claim 1, wherein the system is configured to estimate cannabinoid and/or cannabinoid metabolite amounts.

15. A method of detecting cannabinoid ingestion timing comprising:
taking measurements using a graphene sensor at a first time and at a second time; and
comparing data of the first time with data of the second time and determining a time window of cannabinoid use based on the comparison.

16. The method of claim 15, further comprising providing a voltage stimulus to the graphene sensor and measuring resulting capacitance values of the graphene sensor elements.

17. The method of claim 15, further comprising providing a voltage stimulus to the graphene sensor over a range of voltage values and measuring resulting capacitance values of the graphene sensor elements.

18. The method of claim 15, further comprising estimating cannabinoid and/or cannabinoid metabolite amounts.

19. The method of claim 15, further comprising taking breath samples of a test subject at a first time and at a second time.

20. The method of claim 15, wherein the graphene sensor is a graphene varactor.

21-53. (canceled)
